(19) United States
(12) Patent Application Publication
(10) Pub. No.: US 2010/0047393 A1
(43) Pub. Date: Feb. 25, 2010

(54) PROBIOTIC (INFANT) FOOD

(76) Inventors: Cornelis Glas, Tietjerk (NL); Rob te Biesebeke, Amersfoort (NL); Jeroen Lucas Kiers, Amersfoort (NL); Edward Eelco Salomon Nieuwenhuis, Rotterdam (NL)

Correspondence Address: HOFFMANN & BARON, LLP, 6900 JERICHO TURNPIKE, SYOSSET, NY, 11791, US

(21) Appl. No.: 12/514,374

(22) PCT Filed: Nov. 9, 2007
(86) PCT No.: PCT/NL2007/050549

§ 371 Date: Aug. 13, 2009

(30) Foreign Application Priority Data
Nov. 9, 2006 (NL) 1032840

(51) Int. Cl. A23L 1/29; C12N 1/20

(52) U.S. Cl. 426/61; 435/252.4

(57) ABSTRACT

The invention relates to foods, such as infants and children's food and dietary supplements for infants and children. The invention provides a composition comprising a lipid source, a carbohydrate source and a protein source, characterized in that the composition further contains the B. lactis strain deposited under ATCC number 27536 and the L. casei strain deposited under ATCC number 55544. Such a composition contributes to the general resistance, in particular with respect to undesired passage of pathogens and the incidence of respiratory diseases in children.

The invention relates to food compositions, such as infant food and dietary supplements for children. In particular, the invention relates to formulations which, also with long-term use, are suitable for infants or children who are (hyper)sensitive to components of ruminant milk (such as infants and children with cow's milk allergy) or have an increased risk of developing such a sensitivity. The invention further relates to formulations which make a positive contribution to the general resistance, in particular to airway infections in infants and children.

In the Netherlands, between 2 and 3% of bottle-fed infants develop an allergy to cow's milk protein. A primary cause of the occurrence of cow's milk allergy is a combination of heredity and environmental factors. Children from a family in which allergic diseases such as asthma, atopic eczema, hay fever and/or food allergy occur have a higher risk of developing a cow's milk allergy. Normally, the infant builds up a tolerance to food proteins after birth. It is found that this process of building up does not yet proceed optimally in infants with cow's milk allergy.

In addition to heredity, other factors play a role as well. Thus, the digestive capacity with respect to proteins is not yet optimal in infants. This is caused by a relatively low level of protein-splitting enzymes and a relatively high pH in the stomach. Further, the intestine often still has an increased permeability so that the protein molecules can pass the intestinal wall and are recognized as being foreign after resorption. These factors explain the relatively high prevalence of cow's milk allergy at the infant age.

In addition, it is found that microbial stimulation during the first year of life plays an important role in the development and maturation of the immune system. Non-allergic children have more bifidobacteria and fewer clostridia in their feces than allergic children. It is remarkable that the change in intestinal flora precedes the development of the allergic symptoms, which suggests that the composition of the intestinal flora may affect (the risk of) the development of allergy.

In children, the symptoms of cow's milk allergy are typically manifest in the skin, the gastrointestinal tract and also the airways. None of these symptoms are conclusive for or specific to cow's milk allergy, which makes the diagnostics difficult. The most common symptoms in young children are diarrhea, food problems (such as troubled drinking), vomiting, colics, eczema, urticaria, dyspnea, coughing or wheezing after the intake of food.

Prevention is recommended for infants with a positive family anamnesis: these are infants who have one parent, brother or sister with a proven cow's milk allergy. Up to now, for children with a positive family anamnesis, a diet based on partial or extensive milk protein hydrolysate was advised; the antigenic protein epitopes have been destroyed partially (partial hydrolysate) or virtually completely (extensive hydrolysate) by enzymatic treatment.

Known hydrolysates of cow's milk proteins are whey protein and casein hydrolysates. For use in children with a (hyper)sensitivity to ruminant milk fats, such as for instance Infantile Refsum Disease (IRD) patients, preferably use is made of milk protein-containing starting materials which are low in milk fat, such as casein or caseinate.

Extensively hydrolyzed milk protein products, also referred to as first-generation products, contain mainly peptides with a molecular weight lower than 1500 daltons. It has been found that it is favorable for a maximal reduction of the allergenicity. Foods based on extensive casein hydrolysate have a proven favorable effect on children who are sensitive to ruminant proteins. For instance, applicant published, on the basis of a double-blind study in children with cow's milk allergy, about the safety of such a hypoallergenic product (known under the trade name of Frisolac Allergycare®) (Terheggen-Lagro et al. BMC Pediatrics (2002), 2: 10).

Although the product is safe for the target group, it still has a number of drawbacks which particularly become manifest with long-term use, for instance during at least a few months. Thus, there is a higher concentration of urea in the blood, which is an indication of an overloaded amino acid metabolism. In contrast with fatty acids and glucose, amino acids cannot be stored. Amino acids cannot be secreted either. In the metabolism of proteins (amino acids), carbon dioxide and ammonia are released. The urea cycle produces urea from ammonia and carbon dioxide and allows excretion of excess nitrogen in the form of urea via the urine in a non-toxic and well soluble form.

In addition, an extensive hydrolysate has no or a very low capacity to induce (oral) tolerance to milk proteins (see JP-03-181681; EP 629350; Schmidt et al., Intestinal Immunology and Food Allergy, 34th Nestlé Nutrition Workshop, pp. 24-28). The development of oral tolerance is possibly related to the so-called tight junctions in the intestinal wall closing in delayed manner and/or not closing in time and the associated permeability of the intestinal wall to allergens.

It is important for children in particular, but also for teenagers, adults and elderly people (both men and women) to increase the general resistance by means of a healthy diet. This is particularly the case with respect to the risk of passage of pathogens through the intestinal wall, and with respect to increasing the resistance to occurring gastrointestinal symptoms and respiratory diseases, but also all kinds of infections, such as eye infections, skin infections, ear infections and/or inflammations of the ear, and infections of the skin, the internal and/or external genitals, and the urinary system.

Passage of microorganisms through the intestinal wall is not only understood to mean infections in the blood, also referred to as sepsis, but also infections which can cause meningitis after passage through the intestinal wall, or other types of infections in bodily fluids.

Infections of the gastrointestinal tract comprise gastroenteritis, which is characterized by infections and/or inflammations of the mucosae of the stomach and the intestines, and acute and chronic diarrhea. Acute diarrhea is characterized by a suddenly occurring deviation from the defecation pattern typical for a person with a maximum duration of 14 days, while the frequency and the amount of feces are increased and the feces contain more water than usual. Chronic diarrhea has the symptoms of acute diarrhea, but generally lasts longer than 14 days. Gastrointestinal symptoms are also understood to mean abdominal pain and cramps and specific diseases such as for instance a Helicobacter pylori infection.

Respiratory diseases comprise infections which relate to inflammation of the smaller airways (bronchitis) which are caused by (inter alia) bacterial or viral (inter alia common cold and influenza virus) infection or a combination thereof This is also understood to mean: influenza caused by the influenza type A, B or C: the contagious viral infectious disease, which often occurs as an epidemic. Other examples are severe inflammations of the lung tissue due to various microorganisms, and pneumonitis, a term which is typically used in case of a mild form of pneumonia. These inflammations can often be identified by acute, high fever, pain on the chest, side and/or back, cold shivers, dyspnea, bringing up phlegm, white-bluish skin due to lack of oxygen—also called cyanosis—and nostril breathing. The latter particularly occurs in children. Respiratory diseases are also understood to mean a disease or infection of the trachea, also known as tracheitis. This also includes the constriction of the passage of the airways so that the respiration becomes more difficult and (very severe) dyspnea can occur as a result of infections, obstructions and excess or pulmonary mucus that is difficult to expel. Further, this also includes common cold, which is in fact an inflammation of the mucosa in the nasal cavity and pharynx and/or the paranasal sinuses of the head (maxillary sinuses and sinus cavities) and is particularly caused by the picorna viruses, but also diseases related to CNSLD (Chronic Non-Specific Lung Disease) such as asthma, chronic bronchitis and pulmonary emphysema. Asthma is characterized by the periodical occurrence of periods of dyspnea, with a difficult exhalation and often already manifests itself at a young age. Chronic bronchitis is a disease where regularly symptoms of coughing and/or bringing up mucus occur and with emphysema, the elasticity of the alveolar tissue is reduced. The latter diseases particularly manifest themselves at a later age as a result of air pollution by industry, but also due to frequent infections of the airways and active/passive smoking. In fact, the above means: all locations on or in the body where an imbalance can occur in the harmless microflora naturally present. This imbalance can occur as a result of a change in the microflora naturally present or as a result of contracting an infection from outside the body. This usually involves a disease the body will need to resist immunologically in order to become healthy or free of the infection or imbalance again. Airway diseases are also understood to mean sinusitises. This is an inflammation of the paranasal sinuses, which include the sinus cavities, maxillary sinuses, sphenoidal sinuses and the ethmoid cells between the eyes. Infections as described hereinabove of the airways, the gastrointestinal tract, the skin, eyes, the internal and/or external genitals, the urinary system and the ears can be caused by pathogenic microorganisms of bacterial, viral, parasitic or fungal origin.

The present invention contemplates providing a food, in particular a children's food, where one or more of above-mentioned drawbacks do not occur. The object of the invention is inter alia to provide an optimal food which is suitable for long-term use by children with a hypersensitivity to ruminant milk proteins, or an optimal food for preventive long-term use by infants and children who have an increased risk of developing this sensitivity. Further, the food is preferably suitable for infants and children who are sensitive to ruminant milk fat, such as children with IRD. The object of the invention is more specifically to increase the general resistance in infants, children, teenagers, adults and elderly people. Most particularly, the object of the invention is to reduce, by means of food, the risk of respiratory diseases and other infections as described hereinabove particularly in infants and children but also in teenagers, adults and elderly people.

The applicant has found that the addition of a specific combination of probiotics to food at least partly obviates above-mentioned drawbacks and improves the result with long-term use of the food.

The invention therefore provides a food composition suitable as a food or (therapeutic) dietary supplement for children, comprising a lipid source, a carbohydrate source and a low-allergenic casein hydrolysate with peptides of maximally 3000 daltons, characterized in that the composition further contains the B. lactis strain deposited under ATCC number 27536 and the L. casei strain deposited under ATCC number 55544. The casein hydrolysate is, for instance, a non-allergenic low-fat extensively hydrolyzed casein hydrolysate.

The present inventors have surprisingly found that the unique combination of these B. lactis and L. casei strains in an infant nutrition has a favorable effect on the general resistance, the immune status and the incidence of respiratory diseases in children. In practice, this incidence may range up to 70% or more. More in particular, a reduction was observed in the incidence of airway infections in children who were fed with a food composition (formula) comprising the combination of above-mentioned probiotics compared to a same formulation without probiotics.

Therefore, the invention further provides a food composition comprising the B. lactis strain deposited under ATCC number 27536 and the L. casei strain deposited under ATCC number 55544, while the composition contains a lipid source, a carbohydrate source and a protein source, with the proviso that the protein source is no low-allergenic casein hydrolysate with peptides of maximally 3000 daltons. This is for instance a children's food known per se based on (partially hydrolyzed) cow's milk proteins, such as casein, whey proteins or mixtures thereof, but also based on vegetable proteins, such as proteins from cereals and legumes, which children's food is supplemented with at least the above-mentioned specific combination of probiotics.

So, the invention provides both a hypoallergenic food and a is standard food. Such food compositions are suitable for both preventive and therapeutic use.

The L. paracasei strain ssp. paracasei deposited under ATCC number 55544 is also known under the code CRL 431 of manufacturer Chr. Hansen.

Bifidobacteria are found to be suitable for reducing the ammonia load, in particular Bifidobacterium animalis, ssp. lactis as deposited under ATCC number 27536 (B. lactis 27536). The latter is also known under the code BB12 of manufacturer Chr. Hansen. In vitro analysis shows that the BB12 strain indicates an increase in the expression of the endothelial growth factor B in dendrite cells. The present inventors postulate that this strain is favorable to the improvement or repair of the barrier function of the intestinal wall.

Administering the combination of ATCC 55544 and ATCC 27536 to infants during a period of 12 months resulted in a change in specific T-cell subsets, namely the CD3⁺ and CD3⁺CD4⁺populations.

Without wishing to be bound to any theory, the inventors postulate that the improvement of the resistance to undesired infections observed in children is the result of the combined effect of the probiotic strains L. casei CRL431 and B. lactis BB-12 at the level of the gastrointestinal tract, the mucosal barrier and the airways.

In addition to the probiotic strain which is capable of reducing the ammonia load of the body, the composition may also contain other components which can contribute to the processing of the excess of ammonia, such as the amino acid arginine, which plays an important role in the urea cycle.

The amount of probiotic bacteria in a composition of the invention may vary, and may depend on the type of probiotic and/or the desired properties of the composition in relation to the intended use. Preferably, the composition contains at least 10³, preferably 10³ to 10⁹, such as 10⁶ to 10⁷ colony-forming units (cfu) per gram of a probiotic lactic acid-producing strain. The composition contains, for instance, per gram, 10⁶ to 10⁸ colony-forming units of L. paracasei ATCC-55544 and 10⁶ to 10⁸ colony-forming units of B. lactis ATCC-27536. The relative ratio between the above-mentioned strains is, for instance, between 10:1 and 1:10, preferably between 5:1 and 1:5, such as between 2:1 and 1:2. Good effects were obtained with a mixture of about the same amounts per strain.

A composition according to the invention preferably contains at least one component which has a prebiotic effect on one or more probiotics present in the composition. Examples of suitable prebiotics are fructo and/or galacto-oligosaccharides, with short or long chains, inulin, fucose-containing oligosaccharides, beta glycans, carob flour, gums, pectins, sialyloligosaccharides, sialyllactose, galactans with short or long chains, and nucleotides.

Surprisingly, the low-allergenic protein hydrolysate in a composition according to the invention has also been found to be capable of stimulating the growth of the lactic acid-producing probiotic strains in the intestinal tract. So, the hydrolysate has both a nutritional and a prebiotic role.

Due to the oral intake of a composition with above-mentioned probiotics and the prebiotic effects of peptides from the low-allergenic protein hydrolysate, the imbalance in the intestinal flora of children with an allergy to cow's milk proteins can at least partly be repaired.

The low-allergenic casein hydrolysate is preferably low-fat. It preferably contains mainly peptides having a molecular weight lower than 1500 daltons. Particularly suitable is a hydrolysate which consists for at least about 80 mol % of peptides of maximally 3 amino acids and/or contains no peptides having a length of more than 6 amino acids. The content of peptides smaller than 0.5 dalton is, for instance, 40-60%, while the proportion of free amino acids is preferably as low as possible on account of their bitter taste. Particularly suitable is a hydrolysate having a peptide length distribution as described in Terheggen-Lagro et al. (BMC Pediatrics (2002), 2: 10).

With a view to an optimal closing of the tight junctions and/or the prevention of undesired intestinal permeability (and consequently increased risk of allergy), in addition, one or more non-essential amino acids may be added which provide extra support for this. As described in WO01/58283, NL-1025900 and NL-1027262, in the name of applicant, to this end, inter alia glutamine, glutamic acid, arginine and proline are suitable. Here, glutamic acid is preferred over glutamine since glutamine typically has a reduced stability in products and, after enzymatic hydrolysis in the intestine, can contribute to an undesired ammonia load as well.

As stated hereinabove, an extensively hydrolyzed casein hydrolysate in which allergenic peptides are absent contains no or hardly any tolerance-inducing peptides anymore. In order to wholly or partly ‘repair’ the tolerance-inducing properties, a composition according to the invention may, in addition to the probiotics, also contain a tolerance-inducing milk protein, for instance in the form of a milk protein or whey protein hydrolysate. Tolerance induction typically becomes manifest in the induction and maintenance of a balanced immune system when an organism is loaded with antigenic epitopes. This is, for instance, expressed in the T-cell balance, such as the relative amounts of T-helper cells (Th1, Th2 and Th3 cells).

Preferably, the tolerance-inducing milk protein is a low-milk fat partially hydrolyzed whey protein hydrolysate. In particular, the whey protein hydrolysate is rich in or comes from alpha lactalbumin-rich whey protein. Suitable whey protein hydrolysates have, for instance, a degree of hydrolysis (DH) which may vary between about 5 and 25%. This also results in the amino acid pattern of the composition becoming more similar to that of human milk.

As a carbohydrate source, any type of carbohydrate, or a mixture of different carbohydrates, can serve which is normally used in children's food formulations. Suitable carbohydrate sources are disaccharides such as lactose and saccharose, monosaccharides, such as glucose, and maltodextrins, starch and carbohydrate sources having a prebiotic effect.

The lipid source in a composition according to the invention may be any type of lipid or combination of lipids which are suitable for use in children's food. Examples of suitable lipid sources are tri, di, and monoglycerides, phospholipids, sphingolipids, fatty acids, and esters or salts thereof. The lipids may have an animal, vegetable, microbial or synthetic origin. Of particular interest are polyunsaturated fatty acids such as gamma linolenic acid (GLA), dihomo gamma linolenic acid (DHGLA), arachidonic acid (AA), stearidonic acid (SA), eicosapentaenoic acid (EPA), docosahexaenoic acid (DHA), docosapentaenoic acid (DPA) and conjugated linoleic acid (CLA). CLA is important in the protection against eczema and respiratory diseases in children. This particularly involves the cis-9, trans-11 and cis-12 isomers of CLA. AA may be added to the composition for an optimal tolerance induction.

Further, the composition may contain one or more conventional micro ingredients, such as vitamins, antioxidants, minerals, free amino acids, nucleotides, taurine, carnitine and polyamines. Examples of suitable antioxidants are BHT, ascorbyl palmitate, vitamin E, alpha and beta carotene, lutein, zeaxanthin, lycopene and phospholipids.

In one embodiment, the composition relates to a food or dietary supplement for infants. In a specific form, it relates to a complete infant food up to the age of 6 months. Another specific form relates to a (follow-on) infant food for infants having an age from 6 months. The protein content of the composition is typically less than 3.5 grams per 100 kcal. In another specific case, it relates to a growing-up milk for children in the age group of 1-3 years. In another specific case, it relates to a growing-up milk for children in the age group of 3-6 years. In another specific case, it relates to a dietary milk and/or product for children in the age group of 3-10 years, or a dietary milk and/or product for teenagers in the age group of 10-20 years, for adults of 20-65 years or for elderly people of 65-110 years.

As mentioned hereinabove, the present invention inter alia contemplates providing a hypoallergenic composition which, also in the long term, is suitable for children with a sensitivity to ruminant milk fat. For instance, for children with IRD, for whom long-term use of this milk fat results in accumulation of phytanic acid in the fatty tissue, in particular the nervous tissue.

Phytanic acid in milk fat is a C20 fatty acid with branched chains and comes from chlorophyll, which is taken in by ruminants with the grass or extracted roughage. Due to a disorder in the degradation of phytanic acid which takes place in the peroxisomes, for instance due to absence or inactivity of one or more enzymes and/or absence or a lack of peroxisomes, phytanic acid accumulates in the body.

This problem is particularly known in patients who have Refsum Disease, a peroxisomal metabolic disease. In the infantile form of Refsum Disease (also referred to as “Phytanic Acid Storage Disease” (PASD)), there is a lack of peroxisomes, which causes accumulation of different fatty acids in blood and tissues. The first symptoms are already present at birth. There is an intellectual decline, slightly deviant features, deafness, osteoporosis and retarded growth. Damage to the retina causes nyctalopia and increasingly bad eyesight (retinitis pigmentosa). The liver is enlarged, and the cholesterol content in the blood is elevated (hypercholesterolemia). For Refsum Disease, infantile form, no cure is possible. The treatment of Refsum Disease consists of following a strictly phytanic acid-free diet and is aimed at alleviating symptoms as far as possible.

In a specific embodiment, the invention provides a composition suitable as a food or therapeutic dietary supplement for children who have disorder in phytanic acid metabolism, such as children with the infantile form of Refsum Disease. Such an “IRD composition” comprises a low-phytanic acid lipid source, a carbohydrate source and a non-allergenic casein hydrolysate with peptides of maximally 3000 daltons, characterized in that the composition comprises at least one lactic acid-producing probiotic bacterial strain which, after uptake into the body, can induce the tolerance to milk proteins and/or contains at least one lactic acid-producing probiotic bacterial strain which can reduce the ammonia load. Preferably, the lipid source is phytanic acid-free. The composition contains, for instance, at most 0.1 wt. % of phytanic acid-containing (cow's milk) fat, preferably at most 0.05 wt. %, more preferably at most 0.01 wt. %.

It is has been demonstrated that Refsum patients, in addition to the accumulation of phytanic acid, have a low content of docosahexaenoic acid (DHA) and arachidonic acid (AA) in their circulation Moser et al., Neurochem. Res. 1999, 187-197). Suppletion of DHA and AA through the food, in particular in children, is therefore recommended. Therefore an IRD composition preferably contains DHA and/or AA, more preferably both DHA and AA. DHA and AA are sensitive to oxidation and therefore these long-chain polyunsaturated fatty acids are preferably used in combination with antioxidants in a composition according to the invention.

The invention further provides the use of a food or therapeutic composition according to the invention for treating or preventing symptoms associated with allergy to ruminant milk proteins. The food to be prepared or the composition is particularly suitable for long-term use, preferably during at least a few months.

The symptoms associated with allergy to ruminant milk proteins are very diverse and comprise inter alia a delayed closing of the tight junctions of the intestinal wall, an imbalance in the intestinal flora and/or elevated urea levels in the blood.

Due to its increased nutritional value, a composition according to the invention can advantageously be used for improving the growth of children, in particular as (long-term) therapeutic for children with an allergy to ruminant milk proteins or as (long-term) prophylactic for children who have an increased risk of developing such an allergy.

In addition, due to the use of a composition, the remission percentage of children with (a risk of) allergy can be increased. Remission percentage is understood to mean: allergy staying away with long-term use, expressed as a percentage of the monitored “problem children”. As a reference point for this parameter, it holds that, in practice, the majority of infants fed with mother's milk finally grow out of the allergy, with about 50% in the first year, about 70% in the second year and about 85% in the third year. With a composition according to the invention, the remission percentage after 1 year can be improved by at least 5%, preferably by at least 10%.

A non-allergenic composition according to the invention can have a favorable effect on the ammonia load, the intestinal wall closure, the prevention of adhesion of pathogens to the intestinal wall and/or the repair of the intestinal flora and is therefore particularly suitable for long-term preventive use when children with an allergic family anamnesis are involved, i.e. children with an increased risk of developing an allergy to milk proteins.

Another aspect of the invention provides the use of the combination of the B. lactis strain deposited under ATCC number 27536 and the L. casei strain deposited under ATCC number 55544 for the preparation of a composition for prevention or treatment of respiratory diseases, gastrointestinal infections or symptoms but also eye and/or ear infections but also infections to the skin, the internal and/or external genitals, and the urinary system in a child, in particular a child in the age up to 12 months, such as 1-6 months, but also in children in the age of 1-10 years, teenagers of 10-20 years, adults of 20-65 years and elderly people of 65-110 years. The invention also provides a method for the treatment of a disease, comprising administering a composition comprising an effective amount of a prophylactic or therapeutic composition for treatment of infections, such as a respiratory disease.

The invention further provides a mixture of the B. lactis strain deposited under ATCC number 27536 and the L. casei strain deposited under ATCC number 55544. These strains are, for instance, present in a relative ratio of between 10:1 and 1:10, preferably between 3:1 and 1:3, such as between 2:1 and 1:2, and more preferably about 1:1, expressed in colony-forming units. Optionally, the mixture contains additional (probiotic) microorganisms. The mixture may further comprise an edible carrier substance, preferably a powdery carrier substance such as maltodextrin. As appears from the above, a probiotic mixture of the invention can advantageously be used as a dietary supplement. In a specific embodiment, the invention provides a dietary supplement comprising the B. lactis strain deposited under ATCC number 27536 and the L. casei strain deposited under ATCC number 55544 in a relative ratio of about 1:1, diluted with a powdery carrier substance. A dietary supplement is, for instance, used for the preparation of a food product while, with a normal intake of the product, a total amount of these strains of between 10³ en 10¹⁰ cfu is taken in. This can be used for prevention and treatment of infections in and on the human body.

The invention is illustrated on the basis of the following Examples.

### EXAMPLES

### Example 1

### Stimulation of the Growth of Lactic Acid-Producing Probiotics by Extensively Hydrolyzed Casein Hydrolysate

Starting from a minimal medium for the growth, it was investigated whether suitable lactic acid-producing probiotics were stimulated in their growth by a non-allergenic extensively hydrolyzed casein hydrolysate. The minimal medium comprised per liter:

35 g of lactose, 25 g of sodium acetate, 954 mg of imidazole, 100 mg of L-asparagine, 200 mg of L-cystine, 200 mg of DL-tryptophan, 10 mg of uracil, 10 mg of guanine, 10 mg of adenine, 10 mg of xanthine, 200 mg of alanine, 10 mg of sodium chloride, 200 mg of riboflavin, 200 mg of thiamine HCl, 10 micrograms of folic acid, 600 micrograms of niacin, 1.2 mg of pyridoxamine. 2HCl, 200 micrograms of calcium pantothenate, 10 mg of p-amino benzoic acid, 5 micrograms of biotin, 21 g of L-amino acids Sigma LAA21, 10 ml of salt solution.

The casein hydrolysate was the type of which the molecular weight and the peptide length profile were published in BMC Pediatrics 2002 (2) 10.

For the preferred strains L. paracasei ATCC-55544 and Bifidobacterium lactis ATCC-27536, a clear prebiotic effect on the growth, starting from above-mentioned casein hydrolysate, was determined.

### Example 2

### Basic Prescription for a Food or Therapeutic for Infants and Children Who Suffer from an Allergy or Have an Increased Risk of Developing an Allergy

A food was prepared on the basis of extensively hydrolyzed casein, glucose syrup, maltodextrins, vegetable oils, arginine and micro ingredients, further enriched with the probiotics Lactobacillus paracasei ATCC-55544 and Bifidobacterium lactis (ATCC-27536).

The maximum molecular weight of the peptides in the casein protein hydrolysate was 2.5 kDa. The casein hydrolysate stimulated the growth of both probiotics according to the test according to Example 1.

The food contained 2×10⁷ colony-forming units of both probiotics per gram of powder.

The composition of the powdery probiotic hydrolysate food was per 100 grams of powder (all in grams):

| | |
|---|---|
| Casein hydrolysate | 12 |
| Fat (vegetable) | 27 |
| Glucose syrup + maltodextrins | 55.7 |
| L-Arginine | 0.3 |
| Micro ingredients | 2 |
| Fluid | 3 |

### Example 3

### Results of Test with Infants

A comparative test with the food of Example 2, with and without the added probiotics with infants suffering from allergy, showed the favorable effect of the probiotics on the general resistance, the immune status and the respiratory diseases.

The test comprised 119 infants having an age at the start of the study of 1.4-6 months (average 4.2 months); 55% of the male sex.

The group was randomly divided into a probiotic group and a placebo group. During 12 months, the placebo group was fed with the known infants food Friso 1 Allergy Care (Friesland Nutrition). The probiotics group was fed with Friso 1 Allergy Care supplemented with L. casei CRL431 and B. lactis Bb-12 (10⁷ cfu per strain/gram of formulation).

After 12 months, a marked change was observed of CD3⁺ lymphocytes, determined in favor of the probiotic group. In the placebo group, during a period of 6 months, more respiratory diseases developed compared to the probiotic group.

The food enriched with both cultures can be recommended advantageously and without any particular risks for preventive use in children with an allergic family anamnesis.

### Example 4

### Hypoallergenic Basic Prescription

Prepared were 100 grams of hydrolysate food suitable for IRD children or infants, containing L. paracasei ATCC-55544+B. lactis ATCC-27536, with the following composition (all in grams):

| | |
|---|---|
| Casein hydrolysate | 12 |
| Vegetable + animal + microbial + | 27 |
| synthetic lipids | |
| (containing 0.04% AA + 0.02% DHA + 0.3% CLA) | |
| Cow's milk fat | 0 |
| Glucose syrup + maltodextrins | 55.7 |
| Arginine | 0.3 |
| Micro ingredients | 2 |
| Fluid | 3 |
| Protein equivalent % (calculated; Nx 6.25) | 11.7 |
| Cow's milk fat % (calculated) | <0.06 |

### Example 5

### Preventive Infant Food or Nutrition Based on Milk Proteins

Prepared was a standard formula as stated in Table 1 (Friso 1). The formula contains non-hydrolyzed milk proteins in a weight ratio of casein: whey proteins of 40:60, further fats coming from sources other than milk fat, carbohydrates, prebiotic fibers (GOS), vitamins, minerals and nucleotides. A mixture of probiotics was prepared as stated in Example 3. The probiotics were added to Friso 1 in a concentration as stated in Table 2.

TABLE 1
composition of Friso 1 (per 100 ml)

| Average standard analysis | | Friso 1 |
|---|---|---|
| Protein | G | 1.4 |
| Casein (skimmed milk) | g | 0.56 |
| Whey protein (Deminal90) | g | 0.84 |
| Fat | g | 3.5 |
| Linolenic acid | mg | 435 |
| α-Linolenic acid | mg | 62 |
| DHA | mg | 69 |
| AA | mg | 6.9 |
| Carbohydrates | g | 7.2 |
| Lactose | g | 6.9 |
| Maltodextrin | g | 0.26 |
| Dietary fibers | g | 0.4 |
| Galacto-oligosaccharides | g | 0.4 |
| Minerals | | |
| Calcium | mg | 50 |
| Phosphorus | mg | 30 |
| Iron | mg | 0.78 |
| Copper | μg | 49 |
| Sodium | mg | 20 |
| Potassium | mg | 65 |
| Chlorine | mg | 39 |
| Magnesium | mg | 6.0 |
| Zinc | mg | 0.6 |
| Iodine | μg | 10 |
| Manganese | μg | 33 |
| Selenium | μg | 2.5 |
| Vitamins | | |
| Vitamin A total | μg-RE | 70 |
| Retinol | μg | 58 |
| β-carotene | μg | 40 |
| Vitamin D₃ | μg | 1.2 |
| Vitamin E | mg | 1.3 |
| Vitamin K₁ | μg | 5.1 |
| Vitamin B₁ | μg | 44 |
| Vitamin B₂ | μg | 91 |
| Niacin | mg-NE | 770 |
| Vitamin B₆ | μg | 39 |
| Folic acid | μg | 10 |
| Pantothenic acid | μg | 220 |
| Vitamin B₁₂ | μg | 0.16 |
| Biotin | μg | 1.1 |
| Vitamin C | mg | 9.1 |
| Nucleotides | | |
| AMP | mg | 0.48 |
| CMP | mg | 1.6 |
| GMP | mg | 0.30 |
| IMP | mg | 0.21 |
| UMP | mg | 0.66 |
| Taurine | mg | 6 |
| Choline | mg | 14 |
| L-carnitine | mg | 2.0 |
| Inositol | mg | 3.1 |
| Energy value | kcal | 66 |
| | kJ | 275 |

TABLE 2
Amount of probiotics as added to Friso 1 indicated as
colony-forming units (cfu) per gram of formulation in powdery form.

| Probiotic | |
|---|---|
| L. paracasei CRL431 | 1X10E7 |
| B. animalis lactis | 1X10E7 |

### Example 6

### Dietary Supplement

A mixture was prepared of Lactobacillus paracasei subspecies paracasei CRL431 (approx. 1×10¹³ CFU/g) and Bifidobacterium animalis subspecies lactis BB12 (approx. 1×10¹³ CFU/g) (from CHR Hansen, Copenhagen, Denmark) in a 1:1 ratio. Then the probiotic mixture was diluted (for instance 1:4 but depending on the use) with maltodextrin as a carrier substance or carrier. The thus created probiotic dietary supplement can be added to a food product for humans or animals, in particular to a food product for the human age group of infants of 0-6, 6-12 months, children of 1-3, 3-6 and 1-10 years, teenagers of 10-20 years, adults of 20-65 years and elderly people of 65-110 years, for both men and women.

### Example 7

### Effect of the Probiotics Mixture on the Resistance to Infections

In addition to a breast-fed reference group, from birth, two groups of healthy children were included, with one group getting regular starter food Friso 1 (Alignment prescription) and the other group Friso 1+probiotics. The Friso 1 infants food is composed as described in Table 1. The concentration of probiotics (Lactobacillus paracasei subspecies paracasei CRL431 and Bifidobacterium animalis subspecies lactis BB12) added to Friso 1 is as stated in Table 2. The inclusion was randomized and the intervention was double-blind. The infants were monitored during 6 months from 1 week after birth. During the intervention, antropometric information of the children was collected, the composition of the fecal microflora was examined, as well as the antibiotic use among the children. Further, the nature, magnitude and severity of the infections found in the children were examined.

The probiotics mixture of Lactobacillus paracasei subspecies paracasei CRL431 and Bifidobacterium animalis subspecies lactis BB12 was found to be safe to use and had a positive effect on the infections observed.

1. A composition, preferably an infants food, comprising a lipid source, a carbohydrate source and a low-allergenic casein hydrolysate with peptides of maximally 3000 daltons, characterized in that the composition further contains the B. lactis strain deposited under ATCC number 27536 and the L. casei strain deposited under ATCC number 55544.

2. A composition according to claim 1, wherein the casein hydrolysate is a non-allergenic low-fat extensively hydrolyzed casein hydrolysate.

3. A composition comprising a lipid source, a carbohydrate source and a protein source, characterized in that the composition further comprises the B. lactis strain deposited under ATCC number 27536 and the L. casei strain deposited under ATCC number 55544, and wherein the composition is not a composition according to claim 1.

4. A composition according to claim 3, wherein the protein source is chosen from the group consisting of milk proteins, skimmed milk, caseins, desalinated or non-desalinated whey, whey proteins, (whey) proteins enriched with alpha-lactalbumin, lactoferrin and partially hydrolyzed milk proteins, vegetable proteins and hydrolysates thereof.

5. A composition according to claim 1, wherein the composition contains 10³to 10⁹, preferably minimally 10⁶-10⁷, colony-forming units per bacterial strain per gram of dry product.

6. A composition according to claim 1, wherein the composition comprises a tolerance-inducing milk protein or milk protein hydrolysate.

7. A composition according to claim 6, wherein the milk protein (hydrolysate) is a whey protein (hydrolysate), preferably low-milk fat alpha-lactalbumin hydrolysate.

8. A composition according to claim 1, wherein the composition contains an additional component which contributes to the processing of ammonia, preferably arginine.

9. A composition according to claim 1, wherein the lipid source concerns one or more components chosen from tri, di and monoglycerides, phospholipids and sphingolipids, fatty acids, and esters or salts thereof.

10. A composition according to claim 1, wherein the composition contains at least one polyunsaturated fatty acid, preferably chosen from gamma linolenic acid (GLA), dihomo gamma linolenic acid (DHGLA), arachidonic acid (AA), stearidonic acid (SA), eicosapentaenoic acid (EPA), docosahexaenoic acid (DHA), docosapentaenoic acid (DPA) and conjugated linoleic acid (CLA), more preferably at least DHA and AA.

11. A composition according to claim 1, comprising a prebiotic, preferably chosen from fructo and/or galacto-oligosaccharides, with short or long chains, inulin, fucose-containing oligosaccharides, beta glycans, carob flour, gums, pectins, sialyloligosaccharides, sialyllactose, galactans with short or long chains, and nucleotides.

12. A composition according to claim 1, comprising the combination of at least one Bifidobacterium lactis strain and one or more prebiotics for this strain, preferably chosen from nucleotides, sialyloligosaccharides, fructooligosaccharides (FOS), glucooligosaccharides (GOS) and sialyllactose.

13. A composition according to claim 1, wherein the composition comprises maximally 0.1 wt. % of phytanic acid-containing fat, based on the final composition, preferably maximally 0.05 wt. %, more preferably maximally 0.01 wt. %.

14. The use of a composition comprising a lipid source a carbohydrate source and a low-allergenic casein hydrolysate with peptides of maximally 3000 daltons, characterized in that the composition further contains the B. lactis strain deposited under ATCC number 27536 and the L. casei strain deposited under ATCC number 55544 wherein the composition contains 10³ to 10⁹, preferably minimally 10⁶-10⁷, colony-forming units per bacterial strain per gram of dry product for treating or preventing symptoms associated with allergy for ruminant milk proteins.

15. The use of a composition according to claim 13 for the preparation of a food or therapeutic composition for infants or children with a disorder of the phytanic acid metabolism, preferably for children with Refsum Disease.

16. The use of a composition according to claim 1 as a food or therapeutic composition for treating or preventing respiratory diseases in children.

17. The use of the B. lactis strain deposited under ATCC number 27536 in combination with the L. casei strain deposited under ATCC number 55544 for the preparation of a preventive or therapeutic composition for prevention or treatment of infectious diseases of the gastrointestinal tract, the airways and sinus cavity, the ears or ear cavities, the eyes, the urinary system, the internal and external surfaces of genitals and the skin of people in general, and respiratory diseases in infants and children in particular.

18. Use according to claim 14, wherein the food or the composition is intended for long-term use, preferably during at least 3-6 months.

19. A mixture of the B. lactis strain deposited under ATCC number 27536 and the L. casei strain deposited under ATCC number 55544.

20. A mixture according to claim 19, in which the strains are present in a relative ratio of between 3:1 and 1:3, preferably between 2:1 and 1:2, more preferably about 1:1, expressed in colony-forming units.

21. A mixture according to claim 19, further comprising an edible carrier substance, preferably a powdery carrier substance.

22. Use of a mixture according to claim 19 as a dietary supplement.
